(19) United States
(12) Patent Application Publication
(10) Pub. No.: US 2023/0296214 A1
(43) Pub. Date: Sep. 21, 2023

(54) OPTOELECTRONIC APPARATUS AND METHOD FOR PROJECTING A LIGHT FIELD PATTERN ONTO AN OBJECT USING AN OPTOELECTRONIC APPARATUS

(71) Applicant: ams-OSRAM International GmbH, Regensburg (DE)

(72) Inventors: Joel LALONE, Farmington Hill, MI (US); Kimberly PEILER, Northville, MI (US)

(21) Appl. No.: 17/698,150

(22) Filed: Mar. 18, 2022

(51) Int. Cl. F21S 8/00; F21V 23/00; H05B 45/10

(52) U.S. Cl. CPC F21S 8/006; F21V 23/003; H05B 45/10; F21Y 2115/10

(57) ABSTRACT

The invention concerns an optoelectronic apparatus comprising a light field pattern generating device with at least one light emitting element and at least one imaging optic, and a first sensor device, wherein the first sensor device is configured to determine the position of a body part, in particudefinilar the face, of a user of the optoelectronic apparatus relative to the light field pattern generating device, and wherein the light field pattern generating device is configured to generate at least one light field pattern on the body part of the user that is not directly visible to the user when looking in the direction of the light field pattern generating device, the light field pattern comprising at least a first area with a first illuminance and a second area with a second illuminance smaller than the first illuminance.

The present invention concerns an optoelectronic apparatus with at least one optoelectronic component configured to emit a light field pattern during use of the optoelectronic apparatus as well as a method for projecting a light field pattern onto an object using an optoelectronic apparatus.

### BACKGROUND

Currently there is no way to accurately model the distribution (intensity/spectra over area and time) of natural light as it falls on a body part of a human, in particular the human face, in a natural environment. In addition, there is currently no way to simulate this same distribution and project it onto a body part located in a closed space, such as an autonomous vehicle, for the purposes of wellness and allowing the human/occupant(s) to disengage from the habitual nervous-system stimulation (i.e., fight-or-flight responses) learned while located in the closed space/operating the vehicle.

The object of the invention is thus to counteract at least one of the aforementioned problems and to provide an improved optoelectronic apparatus for projecting a light field pattern onto a body part of a human such as for example the human face. It is a further object of the invention to provide a method for projecting a light field pattern onto a human face using a respective optoelectronic apparatus.

### SUMMARY

This and other requirements are met by an optoelectronic apparatus having the features of claim 1 and a method for projecting a light field pattern onto an object having the features of claim 10. Embodiments and further developments of the invention are described in the dependent claims.

The concept, the inventors propose, is to provide an optoelectronic apparatus configured to project at least one light field pattern, and in particular a time changing light field pattern, onto a body part of a human, the light field pattern imitating natural light as it falls on a body part of a human, in particular at least the face of the human using the optoelectronic apparatus, in a natural environment. The optoelectronic apparatus therefore comprises a light field pattern generating device with at least one light emitting element and at least one imaging optic, to project the at least one light field pattern onto the body part. To ensure that the light field pattern is projected onto the body part even though the body part may move or my change position, a first sensor device is provided configured to determine the position of the body part relative to the light field pattern generating device. The light field pattern may correlate to
a light/shadow distribution falling on a photometry “target” that is the size and shape of a human face captured in a natural environment,
a completely simulated theoretical light/shadow distribution, or
a light/shadow distribution that would potentially fall onto the body part of the human but is hindered from doing so and which is -in unhindered satecaptured by a second sensor device.

The optoelectronic apparatus is configured to generate the at least one light field pattern onto the body part of the user such that the light field pattern is not directly visible to the user when looking in the direction of the light field pattern generating device. In contrast, for example a display “projects” an image into the direction of a user of the display such that the image is visible to the user when looking in the direction of the display. The optoelectronic apparatus however projects the at least one light field pattern onto the body part such that the light field pattern is visible on the body part but not directly when looking in the direction of the light field pattern generating device.

The optoelectronic apparatus can in particular be configured to simulate a light/shadow pattern falling onto a body part and at least the face of a human located in a closed space, such as an autonomous vehicle, for the purposes of wellness and allowing the human/occupant(s) to disengage from the habitual nervous-system stimulation (i.e., fight-or-flight responses) learned while located in the closed space/operating the vehicle.

In one aspect, an optoelectronic apparatus is proposed comprising a light field pattern generating device with at least one light emitting element and at least one imaging optic. The optoelectronic apparatus further comprises a first sensor device, wherein the first sensor device is configured to determine the position of a body part, in particular the face, of a user of the optoelectronic apparatus relative to the light field pattern generating device. The light field pattern generating device, in particular knowing the position of the body part, is configured to generate at least one light field pattern on the body part of the user, wherein the light field pattern is thereby not directly visible to the user when looking in the direction of the light field pattern generating device. The light field pattern comprises at least a first area with a first illuminance and a second area with a second illuminance smaller than the first illuminance, and in particular correlates to a light shadow pattern falling on the body part. The first area with the first illuminance may thereby for example correlate to the brightest area of the at least one light field pattern and the second area with the second illuminance may for example correlate to the darkest area of the at least one light field pattern.

In some aspects, the light field pattern generating device is configured to generate the at least one light field pattern at least onto the face of the user of the optoelectronic apparatus. The light field pattern generating device can however also be configured to generate the at least one light field pattern onto the face and one or more further body parts of the user of the optoelectronic apparatus. It may also be conceivable that the light field pattern generating device is configured to generate different light field patterns onto different body parts of the user of the optoelectronic apparatus.

In some aspects, the first and the second illuminance differ at least by a factor of 2. This factor is however only exemplarily and a factor of at least 5, 10, 15, or greater may also be conceivable. The greater the factor the greater the difference in terms of brightness is between the first and the second area and thus for example between the brightest and the darkest area of the at least one light field pattern. Such a difference may in a natural environment in particular occur between the light and shadow areas of a light/shadow pattern falling on a surface.

In some aspects, the light field pattern generating device is configured to generate the at least one light field pattern on the body part with an illuminance of at least 40.000 lux in the first area. This value is however only exemplarily and it might also be desired, that the light field pattern generating device is configured to generate the at least one light field pattern on the body part with an illuminance of at least 60.000 lux, 100.000 lux, or 130.000 lux in the first area. An illuminance of approximately 40.000 lux can for example correlate to an area in half shadow in natural environment during daylight. An area in bright sunlight in natural environment during daylight can in contrast be illuminated with an illuminance of approximately 130.000 lux and an area in the shadow with an illuminance of approximately 6.000 lux. The light field pattern generating device can on the other hand for example be configured to generate the at least one light field pattern on the body part with an illuminance of down to 0.01 lux in the first and/or second area. Such an illuminance can for example correspond to an illumination of the body part with moonlight. Such an illumination may for example be governed by a rod-dominant scotopic or mesopic eye response curve.

In some aspects, the light field pattern generating device is configured to emit light with a spectrum comparable to the solar spectrum (spectrum of solar radiation in space). In particular the light field pattern generating device can be configured to emit light with a spectrum comparable to the solar spectrum (spectrum of solar radiation on earth) after being reflected from environmental surfaces and/or transmitted through environmental objects (e.g. leaves, clouds, moon, buildings). The light field pattern generating device may therefore be configured to emit not only light in the visible range but also in the infrared and for example the ultraviolet range. The light field pattern generated on the body part can therefore for example not only provide a visible experience but also a feeling experience by locally heating up the body part due to for example an infrared component of the emitted light field pattern. The wellness factor can therefore be increased the closer the light field pattern simulates a natural sunlight/shadow pattern falling on the body part.

In some aspects, the light field pattern imitates natural light falling on the body part comprising at least a light area corresponding to the first area and a shadow area corresponding to the second area. The light field pattern can in particular imitate natural light being reflected from environmental surfaces or transmitted through environmental objects (e.g. leaves, clouds, moon, buildings) falling on the body part at different angles and spatial locations. By projecting such a light field pattern on the body part, a natural environment can be simulated for the human, even though he might be located within a closed space. A wellness factor of the human can therefore be increased, and unwanted habitual nervous-system stimulation learned while located in the closed space can be reduced.

In some aspects, the optoelectronic apparatus further comprises a storage device to store at least one of
a dataset corresponding to a change of the light field pattern over time, the storage device being configured to provide the dataset to the light field pattern generating device to project the time changing light field pattern onto the body part, and
a plurality of datasets each corresponding to a change of different light field patterns over time, the storage device being configured to provide a desired dataset of the plurality of datasets to the light field pattern generating device to project the desired time changing light field pattern onto the body part.

The dataset or the datasets may for example contain sampled data of a sunlight/shadow pattern falling on a surface in natural environment which is then simulated by for example selectively dimming light being projected in the direction of the body part. The key area of interest may therefore for example be the human face, on which the light pattern is projected.

In some aspects, the optoelectronic apparatus further comprises a second sensor device configured to capture a light field pattern on a surface present in the user’s environment, the captured light field pattern comprising at least a first area with a first illuminance and a second area with a second illuminance smaller than the first illuminance. The captured light field pattern may then, in particular in real time, be transferred into a dataset, be provided to the light field pattern generating device, and be projected onto the body part. The optoelectronic apparatus can therefore be configured to reproduce the captured light/shadow distribution falling on a surface present in the user’s environment (through projection) on the body part. This can in particular be advantageous in case of the user of the optoelectronic apparatus being located in a closed space, such as for example a vehicle, to simulate a natural environment, and in particular the natural environment outside the closed space, within the closed space in real time.

The optoelectronic apparatus can therefore for example further comprise a processing device configured to convert the light field pattern captured by the second sensor device into a dataset and provide the dataset to the light field pattern generating device to generate the light field pattern on the body part of the user.

In some aspects, the at least one light emitting element is a simple system/array of discrete LEDs/LED packages or a matrix of LEDs or any pattern of light emitting elements. The at least one imaging optic can be formed by individual projection optics arranged atop each light emitting element or several controllable apertures. The controllable apertures can be used to selectively dim a number of lit areas and thus generate the light field pattern on the body part. The at least one light emitting element can however also be a monolithic array of semiconductor pixels with an array of micro-optics above the pixels serving as the at least one imaging optic. The individual packages or pixels could be either white (to simulate light/shadow patterns) or arrays of different visible spectra recreate the spectrum of illumination in a natural environment. An example could be simulating the spectrum spatially of a natural sunlight spectrum filtered through leaves.

In some aspects, the light field generation device can be configured, to project a light field pattern in a high resolution onto the body part, to even allow a projection of text, fonts, graphics, and animated graphics onto the body part.

It is also proposed a vehicle in particular an autonomous vehicle comprising an optoelectronic apparatus according to at least one of aforementioned aspects. The optoelectronic apparatus could for example be mounted on the vehicle’s “A” pillars or headliner. Additionally, a changing position of the sun, could be simulated using a mobile/moving light field pattern generating device on the same area of the vehicle interior. Using a mobile/moving light field pattern generating device a movement of the body part could in addition be compensated by moving the light field pattern generating device. It is however also conceivable that the light field pattern generating device, and in particular light emitting elements of the light field pattern generating device covers larger areas of the interior of the vehicle or is located on several areas within the vehicle, to simulate a changing position of the sun or to compensate a movement of the body part.

It is also proposed a method for projecting at least one light field pattern onto at least a body part of a user, the method comprising the steps:
Determining the position and/or orientation of the body part, in particular the face, of the user relative to a light field pattern generating device using a first sensor device, the light field pattern generating device comprising at least one light emitting element and at least one imaging optic,
Providing a first dataset to the light field pattern generating device containing information of the at least one light field pattern to be projected on the body part, and
Projecting the at least one light field pattern onto the body part, in particular at least the face, of the user such that the light field pattern is not directly visible to the user when looking in the direction of the light field pattern generating device, the at least one light field pattern comprising at least a first area with a first illuminance and a second area with a second illuminance smaller than the first illuminance.

In some aspects, the step of providing the at least one light field pattern comprises a step of capturing a light field pattern of natural sunlight and shadow falling on a surface in a natural environment. The step of providing the at least one light field pattern may in particular comprise capturing a light field pattern of natural sunlight being reflected from environmental surfaces or transmitted through environmental objects (e.g. leaves, clouds, moon, buildings) resulting in a light and shadow pattern falling on a surface. To this end for example photometry “targets” that are the size and shape of a human face can be placed in a natural environment, with the illuminance and color data of light and shadow falling on the target being measured with an illuminance camera or other spectral detectors at various angles. This is however understood to be an exemplary method of capturing a light field pattern. Other methods to capture a light field pattern are also conceivable. This measured data can then be simulated/converted into a dataset containing information of the captured light field pattern, which can then be provided to the light field pattern generating device to be projected on the body part. The simulation/conversion of the measured data into a dataset containing information of the captured light field pattern may correspond to a distillation of many complex factors into a simple, reproducible pattern of light and shadow in form of the dataset. The advantage of recording actual natural light distributions is that it allows complex patterns of variables to be simplified into an easily reproducible light/shadow pattern. Such an easily reproducible light/shadow pattern can then be projected onto the body part using for example an LED array or multiple LED arrays.

In some aspects, the step of providing the at least one light field pattern comprises capturing a light field pattern on a surface present in the user’s environment using a second sensor device, the captured light field pattern comprising at least a first area with a first illuminance and a second area with a second illuminance smaller than the first illuminance. The captured light field pattern may then, in particular in real time, be simulated/converted into a dataset, be provided to the light field pattern generating device, and be projected onto the body part. Such an alternative can in particular be advantageous to sample, in real time, the light/shadow pattern that is falling on for example the exterior of a vehicle, captured via photosensors and being project, in particular as a distance-corrected duplicate of it, onto the user’s body part.

A first sensor device can be used determining the position and/or orientation of the body part, in particular the face, of the user relative to a light field pattern generating device The position/orientation of the body part and in particular face of the user can in particular be evaluated in real time using IR position sensors, galvanic skin response sensors, or a number of different methods. A software algorithm can calculate the direction/position in which the light field pattern is to be projected such that it falls onto the body part.

Using the sensor device to determine the position and/or orientation of the body part in real time, the step of projecting the at least one light field pattern onto the body part can also performed when the body part moves. It can thus be provided that the light pattern is projected onto the body part even though the body part moves (for example the user turns his/her head).

In some aspects, the method further comprises a step of determining a mood of the user and providing a corresponding dataset to the light field pattern generating device containing information of a light field pattern associated with the mood to be projected on the body part. It can thus for example be determined according to the mood of the user if and if yes which light field pattern is to be projected on the body part. The first sensor device can for example comprise a camera and a software algorithm that first senses the user’s face, and then derives a status of certain variables such as alertness, mood, etc..

The step of projecting the at least one light field pattern onto the body part of the user can for example comprise a projection of a spatial distribution of both the illuminance value and the spectrum in different areas of the light field pattern. The light distribution thereby mimics a realistic spatial and spectral distribution.

An optoelectronic apparatus according to some aspects of the invention could for example be used in the following contexts:
In a static (non-vehicular) setting, such as any human-occupied space without access to natural or exterior light.
In the medical field, specifically for pre-surgical waiting or post surgical recovery. In this application, the apparatus’s impacts on biopotentials could be actively measured and corrected to assist palliative care.
In line or queue situations, to make long wait-times more acceptable. In this application, the projected light field pattern could be different in different points in the line. For example a greater soothing or calming effect could be produced at the rear of the line, gradually diminishing toward the front.

### BRIEF DESCRIPTION OF THE DRAWINGS

In the following, embodiments of the invention will be explained in more detail with reference to the accompanying drawings. It is shown schematically in

FIG. 1 steps of a method for projecting at least one light field pattern onto a bod part of a user according to some aspects of the invention,

FIG. 2A a step of capturing a light field pattern according to some aspects of the invention,

FIG. 2B a step of projecting at least one light field pattern onto the body part of the user using an optoelectronic apparatus according to some aspects of the invention,

FIG. 3 an embodiment of an optoelectronic apparatus to project at least one light field pattern onto a body part of a user of the optoelectronic apparatus according to some aspects of the invention, and

FIG. 4 further steps of a method for projecting at least one light field pattern onto a bod part of a user according to some aspects of the invention.

### DETAILED DESCRIPTION

The present disclosure will now be described more fully hereinafter with reference to the accompanying drawings, in which exemplary embodiments of the disclosure are shown. The disclosure may, however, be embodied in many different forms and should not be construed as limited to the embodiments set forth herein; rather, these embodiments are provided for thoroughness and completeness. Like reference characters refer to like elements throughout the description. The drawings are not necessarily to scale and certain features may be exaggerated in order to better illustrate and explain the exemplary embodiments of the present disclosure.

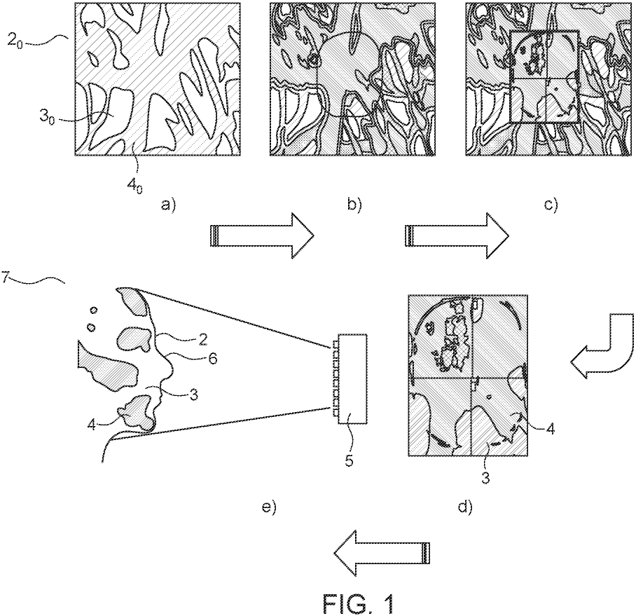

FIG. 1 shows in its subfigures a) to e) schematically steps of a method for projecting at least one light field pattern onto a bod part of in user using an optoelectronic apprartus. In a first step, shown in. subfigure a), of a light/shadow pattern 2₀ falling onto a surface in natural environment is captured using a camera or a colorimeter. This captured raw image may comprise a sun/shadow distribution as it occurs in natural environment depending on the place, the time of day, etc.. The raw image comprises light areas 3₀ as well as shadow areas 4₀ located next to each other which is in a following step, shown in subfigure b), simulated/converted into a false-color luminance image. As indicated in subfigure b) by the oval a photometry “target” that is the size and shape of a human face may be the key area of interest, how the light/shadow pattern falls onto this area.

The illuminance data of the false-color luminance image within this area of interest is then in a further step fully processed, quantified, and split into cells/pixels (in this case quadrants), to be later projected onto a bod part of a user using an optoelectronic apparatus. The illuminance values can thereby be quantified as values with the unit lux, or candela per square meter respectively. The fully processed and quantified image of the area of interest, as shown in subfigure d), is converted into a dataset comprising information of the illuminance values of the cells/pixels (in this case quadrants), to be projected in form of a light field pattern onto the bod part of the user. Former light areas 3₀ are thereby associated with a high illuminance value forming first areas 3 and former shadow areas 4₀ are associated with a low illuminance value forming second areas 4.

The light field pattern 2 is then, as shown in subfigure e), projected onto the body part 6 of the user 7, in this case the face of the user, using a light field pattern generating device 5. The processed luminance data is therefore mapped by the light field pattern generating device 5, which can for example comprise a monolithic array of semiconductor pixels or a discrete LED array as well as at least one imaging optic in form of for example apertures to generate the light field pattern 2 on the body part 6. The light field pattern 2 comprises first areas 3 with a higher illuminance and second areas 4 with a lower illuminance correlating to the captured light/shadow pattern 2₀ falling onto a surface in natural environment.

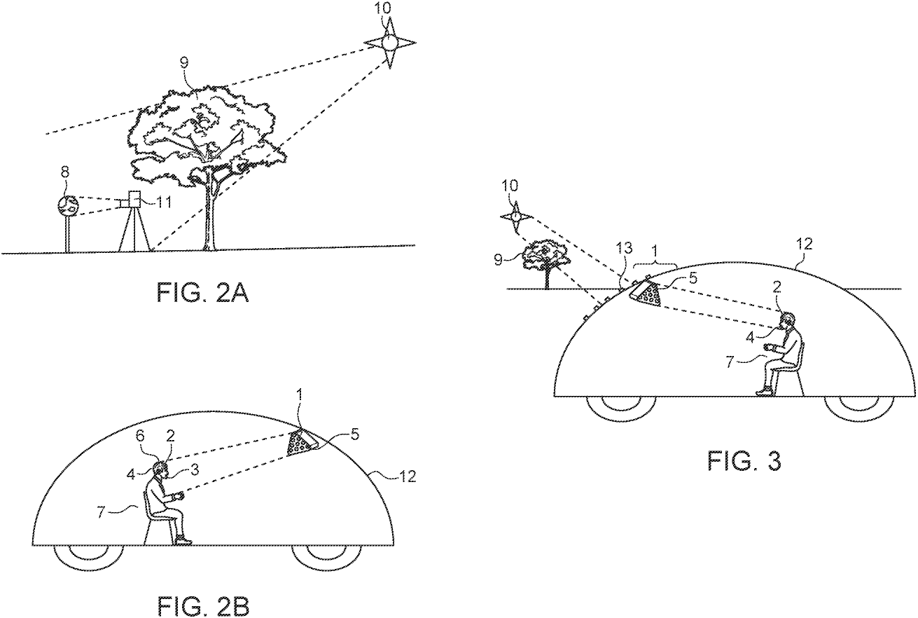

FIG. 2A shows a step of capturing a light/shadow pattern 2₀ as shown in subfigure a of FIG. 1 falling onto a surface in natural environment using a camera or a colorimeter. A photometry “target” 8 that is the size and shape of a human face/head is therefore for example placed in a natural environment. In the shown example the target 8 is placed in the shadow/half shadow of a tree 9 while daylight and the sun 10 is thus shining. Due to the sun shining through the tree crown, a natural light/shadow pattern 2₀ is generated on the target 8 which is then captured using a camera or a colorimeter 11. The captured image can then serve as a basis for a light pattern to be projected onto a body part of a user. Using such a method a plurality of different natural light/shadow patterns at different locations can be captured and converted into a dataset. These datasets can in a later then be used to project several different light field patterns onto a body part of a user of an optoelectronic apparatus depending on the desired need.

FIG. 2B shows an application of an optoelectronic apparatus 1 in use and a step of projecting a light field pattern onto a body part of a user of an optoelectronic apparatus 1 respectively. The optoelectronic apparatus 1 is thereby arranged inside a vehicle 12, in particular an autonomous vehicle. Inside the vehicle, an occupant/user 7 of the optoelectronic apparatus is located driving the vehicle 12. In case of autonomous vehicle, the user 7 may for example not need to pay attention to road traffic or other influencing factors outside the vehicle 12. To divert his/her attention from it, and to contribute to the relaxation of the user 7 the optoelectronic apparatus 1 may project a light field pattern 2 onto the user’s face imitating a natural light/shadow pattern falling on his/her face. The optoelectronic apparatus 1 therefore comprises a not shown first sensor device being configured to determine the position of the face of the user. With this information a light field pattern generating device 5 can project a light field pattern 2 directly onto the user’s face. As soon as the user moves his/her head, the first sensor device would determine the new position of the face in real time and the light field pattern 2 would be projected directly onto the face of the user in the new position.

FIG. 3 shows another embodiment of an optoelectronic apparatus 1. Compared to FIG. 2B, the light/shadow pattern is not captured “in advance” and then projected onto the body part as chosen by the user 7 or the optoelectronic apparatus 1 but captured by a second sensor device 13 located on the outer surface of the vehicle 12 in real time and hence projected onto the body part 4 in real time. Such an alternative can in particular be advantageous to sample, in real time, a light/shadow pattern that is falling on the exterior of the vehicle, capture it via photosensors, and project it, in particular as a distance-corrected duplicate of it, onto the user’s body part.

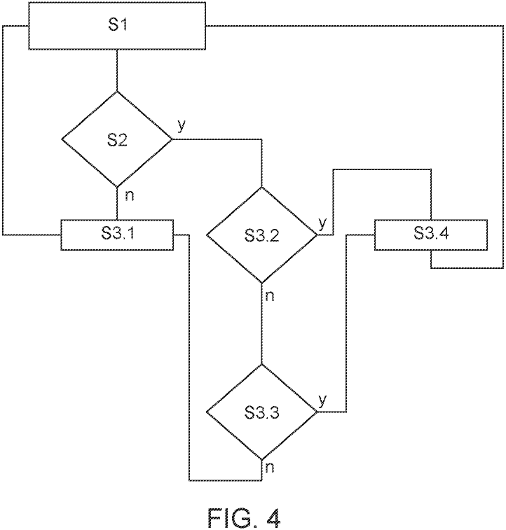

FIG. 4 shows further steps of a method for projecting a light field pattern onto a bod part of a user. FIG. 4 shows in particular a possible software algorithm to determine if a light field pattern shall be projected onto a body part of a user of an optoelectronic apparatus, and in addition determine the mood of the user and choosing a light field pattern of a plurality of light field patterns associated with the mood to be projected onto the body part. A first step S1 correlates to sampling the body part, in particular the face of a user of the optoelectronic apparatus. By this, a mood of the user is determined. Depending on the mood, in a step S2 it is chosen if a treatment of the user in form of projecting a light field pattern onto the body part of the user is activated or not. If yes (y) step S3.2 follows, if no (n), step S3.1 follows and no treatment is activated. In step S3.2 it is checked, if a light field pattern associated with the mood of the user is available. If yes (y) step S3.4 follows, if no (n), step S3.3 follows. In step S3.3 the user is asked if he/she prefers to receive a different treatment. If yes (y) step S3.4 follows, if no (n), step S3.1 follows and no treatment is activated. In step S3.4 a treatment is activated and a light pattern is projected onto the body part. Both step S3.1 and S3.4 are again followed by step S1 again sampling the body part and determining position and/or mood of the user to continue the loop.

LIST OF REFERENCES

| | |
|---|---|
| 1 | optoelectronic apparatus |
| 2 ₀ | light/shadow pattern |
| 2 | light field pattern |
| 3 ₀ | light area |
| 3 | first area with first illuminance |
| 4 ₀ | shadow area |
| 4 | second area with second illuminance |
| 5 | light field pattern generating device |
| 6 | body part |
| 7 | user |
| 8 | target |
| 9 | object |
| 10 | sun |
| 11 | camera |
| 12 | vehicle |
| 13 | sensor device |

What is claimed is:

1. An optoelectronic apparatus comprising
a light field pattern generating device with at least one light emitting element and at least one imaging optic, and
a first sensor device,
wherein the first sensor device is configured to determine the position of a body part, in particular the face, of a user of the optoelectronic apparatus relative to the light field pattern generating device, and
wherein the light field pattern generating device is configured to generate at least one light field pattern on the body part of the user that is not directly visible to the user when looking in the direction of the light field pattern generating device, the light field pattern comprising at least a first area with a first illuminance and a second area with a second illuminance smaller than the first illuminance.

2. The optoelectronic apparatus according to claim 1, wherein the first and the second illuminance differ at least by a factor of 2.

3. The optoelectronic apparatus according to claim 1, wherein the light field pattern generating device is configured to generate the light field pattern on the body part with an illuminance of at least 40.000 lux in the first area.

4. The optoelectronic apparatus according to claim 1, wherein the light field pattern generating device is configured to emit light with a spectrum comparable to the solar spectrum filtered or reflected from environmental surfaces or environmental objects.

5. The optoelectronic apparatus according to claim 1, wherein the light field pattern imitates natural light reflected from environmental surfaces or transmitted through environmental objects falling on the body part comprising at least a light area corresponding to the first area and a shadow area corresponding to the second area.

6. The optoelectronic apparatus according to claim 1, further comprising a storage device to store at least one of a dataset corresponding to a change of the light field pattern over time, the storage device being configured to provide the dataset to the light field pattern generating device; and a plurality of datasets each corresponding to a change of different light field patterns over time, the storage device being configured to provide a desired dataset of the plurality of datasets to the light field pattern generating device.

7. The optoelectronic apparatus according to claim 1, further comprising a second sensor device configured to capture a light field pattern on a surface present in the user’s environment, the captured light field pattern comprising at least a first area with a first illuminance and a second area with a second illuminance smaller than the first illuminance.

8. The optoelectronic apparatus according to claim 7, further comprising a processing device configured to convert the light field pattern captured by the second sensor device into a dataset and provide the dataset to the light field pattern generating device to generate the light field pattern on the body part of the user.

9. A vehicle in particular autonomous vehicle comprising an optoelectronic apparatus according to claim 1.

10. A method for projecting at least one light field pattern onto a body part of a user, the method comprising the steps:
determining the position and/or orientation of the body part, in particular the face, of the user relative to a light field pattern generating device using a first sensor device, the light field pattern generating device comprising at least one light emitting element and at least one imaging optic;
providing a first dataset to the light field pattern generating device containing information of the at least one light field pattern to be projected on the body part; and
projecting the at least one light field pattern onto the body part of the user such that the light field pattern is not directly visible to the user when looking in the direction of the light field pattern generating device, the at least one light field pattern comprising at least a first area with a first illuminance and a second area with a second illuminance smaller than the first illuminance.

11. The method according to claim 10, wherein the step of providing the at least one light field pattern comprises capturing a light field pattern of natural sunlight and shadow falling on a surface.

12. The method according to claim 10, wherein the step of providing the at least one light field pattern comprises capturing a light field pattern on a surface present in the user’s environment using a second sensor device, the captured light field pattern comprising at least a first area with a first illuminance and a second area with a second illuminance smaller than the first illuminance.

13. The method according to claim 12, wherein the step of providing the at least one light field pattern comprises a conversion of the captured light field pattern into a dataset to be provided to light field pattern generating device.

14. The method according to claim 10, wherein the step of projecting the at least one light field pattern onto the body part is also performed when the body part moves.

15. The method according to claim 10, further comprising a step of determining a mood of the user and providing a corresponding dataset to the light field pattern generating device containing information of at least one light field pattern to be projected on the body part associated with the mood.
